(19) United States
(12) Patent Application Publication
(10) Pub. No.: US 2026/0158114 A1
(43) Pub. Date: Jun. 11, 2026

(54) Method for Treating Skeletal Articulations and Composition and Uses Thereof

(71) Applicant: Lonza Greenwood LLC, Greenwood, SC (US)

(72) Inventors: Vijaya Juturu, North Bruswick, NJ (US); Zainulabedin Saiyed, Benicia, CA (US); Shane Durkee, Morristown, NJ (US)

(21) Appl. No.: 18/707,629

(22) PCT Filed: Nov. 7, 2022
(86) PCT No.: PCT/US2022/049073

§ 371 Date: May 6, 2024

Related U.S. Application Data
(60) Provisional application No. 63/278,860, filed on Nov. 12, 2021.

(51) Int. Cl. A61K 38/39; A61P 19/02; A61P 29/00

(52) U.S. Cl. CPC A61K 38/39; A61P 19/02; A61P 29/00

(57) ABSTRACT

An joint treating composition is disclosed that, when administered to mammals in a therapeutically effective amount, can reduce inflammation and even heal injured or diseased skeletal articulations. For example, the joint treating composition is well suited for treating osteoarthritis and other skeletal ailments or injuries. The composition of the present disclosure, for instance, can dramatically decrease various biomarkers related to skeletal health, including reducing tumor necrosis factor serum and protein in the body.

### CROSS REFERENCE TO RELATED APPLICATIONS

This application claims priority to the benefit of U.S. Provisional Application No. 63/278,860, filed Nov. 12, 2021, which is expressly incorporated herein by reference in its entirety.

### BACKGROUND

The matrix of cartilage is comprised of collagens, proteoglycans, and non-collagenous proteins and serves as the cushion and shock absorber within skeletal articulations as it lines the ends of the two bones that form the joint. Cartilage damage can be caused by several conditions including: joint injury, avascular necrosis, the effects of aging, osteoarthritis, auto immune disorder, and rheumatoid arthritis. The damaged cartilage causes pain and can limit the motion of the joint.

Inflammation is a response of a tissue to injury and is characterized by increased blood flow to the tissue causing increased temperature, redness, swelling, and pain. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

One joint health factor that indicates the onset of poor joint health caused by injury or osteoarthritis is the level of tumor necrosis factor alpha (TNF a). TNF a is produced by monocytes, macrophages, and T lymphocytes. TNF a exists as both a soluble form, solTNF, which is believed to play an important role in inflammation, and a transmembrane form, tmTNF, which is involved in immune functions.

TNF a exerts its primary effects on monocytes, synovial macrophages, fibroblasts, chondrocytes, and endothelial cells, and stimulates proinflammatory cytokine and chemokine synthesis. It activates granulocytes and increases MHC Class II expression. It promotes secretion of matrix metalloproteinases (MMPs), leading to cartilage matrix degradation, which indicates inflammation.

Because it initiates an inflammatory cascade and has been found to be increased in close proximity to inflamed or injured tissue, TNF a inhibition is a target for pain and/or inflammation therapy and/or tissue destruction.

Various methods exist for treating skeletal articulations that suffer from injury or disease, such as osteoarthritis. Most of these treatment methods are directed to controlling pain and inflammation. What is needed is a composition that can be administered to mammals for not only treating the pain associated with poor joint health, but also capable of repairing damaged tissue for regaining mobility.

### SUMMARY

The present disclosure is generally directed to a method of improving one or more of joint pain, joint health, joint mobility, or inflammation in mammals. The method includes administering a nutraceutical supplement to the mammal, where the supplement includes a Type II collagen composition. In one aspect, the Type II collagen composition includes undenatured collagen, which has been found to be particularly effective. The Type II collagen is administered to the mammal in an amount sufficient to counteract the effects of a disease or inflammation of a skeletal articulation, such as osteoarthritis or other articulo-necrosis. It was unexpectedly discovered that increasing the dose of the Type II collagen translates into greater joint health efficacy.

In one embodiment, for instance, the present disclosure is directed to a method of treating a skeletal articulation injury or disease in a mammal. The method includes supplying to the mammal a therapeutically effective amount of a joint healing composition. The joint treating composition comprises collagen and can be administered to the mammal in the form of doses. More particularly, the joint treating composition is administered to the mammal in amounts greater than about 0.66 mg/kg of body weight per day (which translated to a human equivalent dose of 40 mg).

In the past, it was widely believed that daily undenatured (or native) collagen doses of greater than about 0.55, such as greater than about 0.6 mg/kg of body weight per day (e.g. 40 mg of collagen for some mammals) would have no added beneficial effect on the user or mammal. It was surprisingly and unexpectedly discovered, however, that administering greater than about 0.66 mg/kg of body weight per day of collagen, particularly undenatured collagen, can have dramatic beneficial effects on joint injuries or diseases. In this regard, the method of the present disclosure, in one aspect, can administer greater amounts of collagen to a mammal sufficient to lessen the severity of a skeletal articulation injury or disease as shown through a change in at least one joint health marker in comparison to administering less than 0.3 mg/kg of body weight per day of the joint treating composition, such as in comparison to administering less than 0.5 mg/kg of body weight per day of the joint treating composition, such as in comparison to administering less than 0.6 mg/kg of body weight per day of the joint treating composition.

For example, the joint treating composition can be administered to the mammal such that the mammal receives collagen, particularly undenatured Type II collagen, in an amount greater than about 1 mg/kg of body weight per day, such as greater than about 2 mg/kg of body weight per day. Collagen can be administered to the mammal, for instance, in an amount from about 1.2 mg/kg of body weight per day to about 3 mg/kg of body weight per day.

The amount of collagen administered to the mammal can depend upon various factors including the type and amount of inflammation and the type of mammal. In one embodiment, the mammal, which can be a human, is administered collagen in an amount greater than about 50 mg per day, such as greater than about 80 mg per day, such as greater than about 100 mg per day, such as greater than about 120 mg per day, such as in an amount greater than about 160 mg per day, such as in an amount greater than about 320 mg per day, such as in an amount greater than about 400 mg per day, such as in an amount greater than about 480 mg per day, and generally in an amount less than about 3,000 mg per day, such as in an amount less than about 1,000 mg per day.

The joint treating composition can be administered to the mammal in an amount sufficient and over a period of time in order to treat inflammation of the skeletal joints and/or treat a joint injury or disease, such as osteoarthritis. The effects of the composition of the present disclosure can be seen in improved mobility and/or by monitoring various biomarkers that are related to inflammation, joint metabolism and/or joint health. For example, the joint treating composition of the present disclosure can be administered to a mammal over a period of time sufficient to decrease serum tumor necrosis factor alpha in an amount greater than about 5%, such as in an amount greater than about 15%, such as in an amount greater than about 25%. Serum C-reactive protein can be reduced by greater than about 10%, such as greater than about 20%, such as greater than about 30%, such as greater than about 40%. Prostaglandin E2 can be decreased by greater than about 8%, such as greater than about 15%, such as greater than about 25%, such as greater than about 30%. Interleukin-1 serum levels can be decreased by greater than about 10%, such as greater than about 20%, such as greater than about 30%, such as greater than about 40%. Interleukin-6 serum can be decreased by greater than about 10%, such as greater than about 20%, such as greater than about 30%, such as greater than about 40%. Interleukin-1-B protein can be reduced by greater than about 8%, such as greater than about 10%, such as greater than about 15%, such as greater than about 20%. Interleukin-6 protein can be decreased by greater than about 10%, such as greater than about 15%, such as greater than about 20%, such as greater than about 30%, such as greater than about 40%. Interleukin-10 protein can be decreased by greater than about 10%, such as greater than about 20%, such as greater than about 30%, such as greater than about 40%. Tumor necrosis factor alpha protein can be decreased by greater than about 10%, such as greater than about 20%, such as greater than about 25%, such as greater than about 35%. Cyclooxygenase 2 protein can be decreased by greater than about 7%, such as greater than about 10%, such as greater than about 20%, such as greater than about 30%. Nuclear factor kappa B protein can be decreased by greater than about 10%, such as greater than about 20%, such as greater than about 30%, such as greater than about 35%. Matrix metallopeptidase 3 can be decreased by greater than about 15%, such as greater than about 25%, such as greater than about 35%. Cartilage oligomeric matrix serum can be decreased by greater than about 10%, such as greater than about 20%, such as greater than about 30%. Cartilage oligomeric matrix protein can be decreased by greater than about 10%, such as greater than about 15%, such as greater than about 25%, such as greater than about 30%. Transforming growth factor B, on the other hand, can be increased by greater than about 5%, such as greater than about 10%, such as greater than about 15%, such as greater than about 20%.

The joint treating composition of the present disclosure can be administered to a mammal over a period of time sufficient to change a Kellgren-Lawrence score of the mammal by at least about 20%. The joint treating composition of the present disclosure can also be administered to a mammal over a period of time sufficient to change a Mankin score of the mammal by at least about 15%.

The above decreases and increases in the above biomarkers can be in comparison to the initial levels of the mammal being treated. The above influences on the biomarkers can occur over a period of time after administering the joint treating composition. The period of time, for instance, can be from about 10 days to about 60 days, such as from about 20 days to about 40 days. In one embodiment, the above effects are realized after a 28-day period. The joint health composition can be administered in any suitable form. In one aspect, the joint health composition is a nutraceutical supplement intended to be ingested. Such forms include solids, liquids, and gels, including capsules, tablets, and powders, etc. The joint health composition can also be in a lipid multiparticulate form (LMP), whether spray dried, in beadlets, or the like.

Other features and aspects of the present disclosure are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a graphical representation of some of the results obtained in the example below;

FIG. 2 is a graphical representation of some of the results obtained in the example below;

FIG. 3 is a graphical representation of some of the results obtained in the example below;

FIG. 4 is a graphical representation of some of the results obtained in the example below;

FIG. 5 is a graphical representation of some of the results obtained in the example below;

FIG. 6 is a graphical representation of some of the results obtained in the example below;

FIG. 7 is a graphical representation of some of the results obtained in the example below;

FIG. 8 is a graphical representation of some of the results obtained in the example below;

FIG. 9 is a graphical representation of some of the results obtained in the example below;

FIG. 10 is a graphical representation of some of the results obtained in the example below;

FIG. 11 is a graphical representation of some of the results obtained in the example below;

FIG. 12 is a graphical representation of some of the results obtained in the example below;

FIG. 13 is a graphical representation of some of the results obtained in the example below;

FIG. 14 is a graphical representation of some of the results obtained in the example below;

FIG. 15 is a graphical representation of some of the results obtained in the example below;

FIG. 16 is a graphical representation of some of the results obtained in the example below;

FIG. 17 is a graphical representation of some of the results obtained in the example below;

FIG. 18 is a graphical representation of some of the results obtained in the example below;

FIG. 19 is a graphical representation of some of the results obtained in the example below;

FIG. 20 is a graphical representation of some of the results obtained in the example below;

FIG. 21 is a graphical representation of some of the results obtained in the example below;

FIG. 22 is a graphical representation of some of the results obtained in the example below;

FIG. 23 is a graphical representation of some of the results obtained in the example below;

FIG. 24 is a graphical representation of some of the results obtained in the example below;

FIG. 25 is a graphical representation of some of the results obtained in the example below;

FIG. 26 is a graphical representation of some of the results obtained in the example below;

FIG. 27 is a graphical representation of some of the results obtained in the example below;

FIG. 28 is a graphical representation of some of the results obtained in the example below;

FIG. 29 is a graphical representation of some of the results obtained in the example below;

FIG. 30 is a graphical representation of some of the results obtained in the example below;

FIG. 31 is a graphical representation of some of the results obtained in the example below;

FIG. 32 is a graphical representation of some of the results obtained in the example below;

FIG. 33 is a graphical representation of some of the results obtained in the example below;

FIG. 34 is a graphical representation of some of the results obtained in the example below;

FIG. 35 is a graphical representation of some of the results obtained in the example below;

FIG. 36 is a graphical representation of some of the results obtained in the example below;

FIG. 37 is a graphical representation of some of the results obtained in the example below;

FIG. 38 is a graphical representation of some of the results obtained in the example below;

FIG. 39 is a graphical representation of some of the results obtained in the example below;

FIG. 40 is a graphical representation of some of the results obtained in the example below;

FIG. 41 is a graphical representation of some of the results obtained in the example below;

FIG. 42 is a graphical representation of some of the results obtained in the example below;

FIG. 43 is a graphical representation of some of the results obtained in the example below; and

FIG. 44 is a graphical representation of some of the results obtained in the example below.

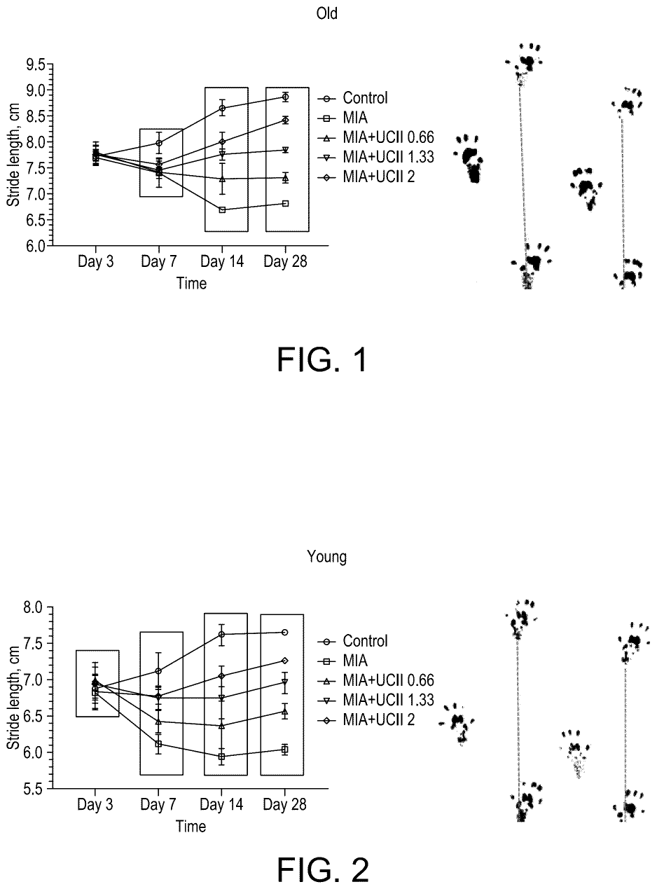

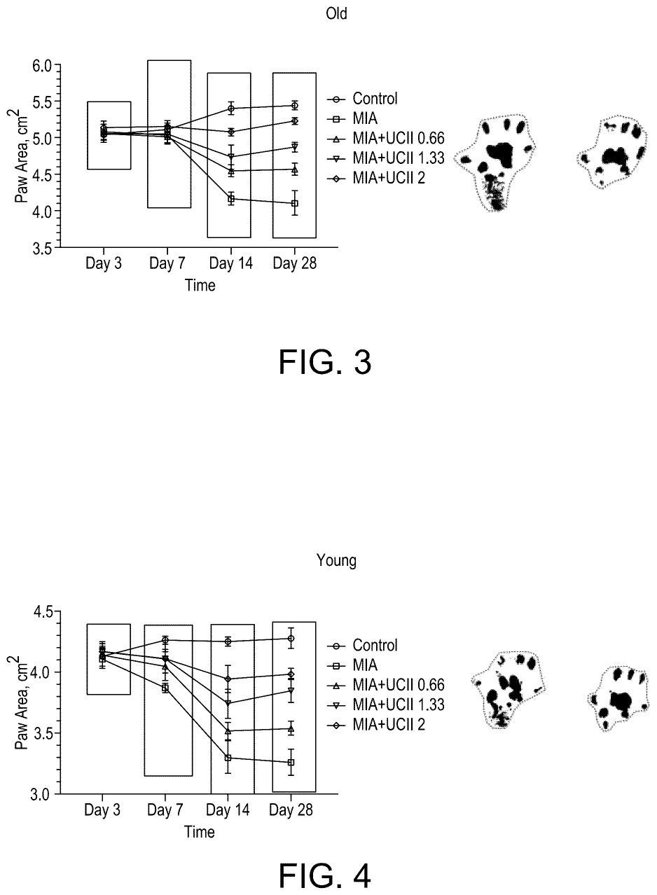

Repeat use of reference characters in the present specification and drawings is intended to represent same or analogous features or elements of the invention.

### Definitions

As used herein, the terms “about,” “approximately,” or “generally,” when used to modify a value, indicates that the value can be raised or lowered by 10%, in one aspect, such as 8%, such as 5%, such as 4%, such as 3%, such as 2%, such as 1% in one aspect, and remain within the disclosed aspect.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation. In one aspect, a therapeutically effective amount refers to supplying or ingesting collagen in an amount to maintain, repair, or improve the health of a joint(s).

As used herein, by “active” or “active ingredient” is meant a drug, medicament, pharmaceutical, therapeutic agent, nutraceutical, or other compound that may be desired to be administered to the body. The active ingredient may be a “small molecule,” generally having a molecular weight of 2000 Daltons or less. The active ingredient may also be a “biological active.” Biological active ingredients include proteins, antibodies, antibody fragments, peptides, oligonucleotides, vaccines, and various derivatives of such materials. In one embodiment, the active ingredient is a small molecule. In another embodiment, the active ingredient is a biological active. In still another embodiment, the active ingredient is a mixture of a small molecule and a biological active. Also as used herein, the terms “active ingredient”, “first active ingredient”, “second active ingredient”, etc. may be used to denote active ingredients located in different places within the particle, such as those located in the core or those located in the one or more outer layers. However, the terms “first” or “second” do not necessarily denote that the first active ingredient is different from the second active ingredient. For example, in certain embodiments, the active ingredient contained within the core may be the same as the second active ingredient contained within an outer layer disposed on the core. While in certain other embodiments, the active ingredient contained within the core may be different from the second active ingredient contained within an outer layer disposed on the core.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation canine, equine, feline, bovine, ovine, human, or porcine mammals.

As used herein, “healthy” refers to the absence of illness or injury.

Unless otherwise noted, “collagen” as used herein refers to all forms of collagen, either with or without denaturation, without or without salts or stabilizing agents, and fibrillar and non-fibrillar types of collagen not limited to fibril associated collagens with interrupted triple helices (FACIT, Type IX, XII, XIV, XIX, XXI), including short chain collagen (generally Types VII and X), basement membrane (Type IV), Multiplexin (multiple triple helix domains with interruptions (Type XV, XVIII), and other types of collagen (Types VI, VII). The collagen, in one aspect, can be maintained with a triple helix structure and with epitopes without any chemical or enzyme or heat or lower pH treatments.

As used herein, the term “biomarker” refers to a measurable substance in a mammal whose presence, concentration or amount is indicative of some phenomenon such as disease, infection or inflammation and can be a joint biomarker an inflammation biomarker or the like.

As used herein, percent change in any one or more of the discussed biomarkers is based upon a baseline value for the respective subject and/or marker and does not refer to a percent change from a midpoint or non-starting point. Therefore, as used herein, “baseline” refers to a day zero or starting point, and can therefore be used to determine a value of a measured marker at a starting point, which is then used to calculate the percent change in any of the biomarkers discussed herein from the baseline.

Other features and aspects of the present disclosure are discussed in greater detail below.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a method for reducing inflammation, increasing mobility and/or otherwise treating diseases or injuries of skeletal articulations in mammals. The method includes administering to mammals, such as animals and humans, affected as described above with a therapeutically effective amount of a joint treating composition and for a period of time sufficient for the health of the mammal and the skeletal articulations to improve. The method of the present disclosure, for instance, is particularly well suited to treating osteoarthritis and/or skeletal necrosis.

The joint treating composition of the present disclosure contains collagen, such as any suitable collagen in various forms. In one embodiment, the collagen contained in the joint treating composition is an undenatured Type II collagen. Collagen has been used in the past as a supplement to improve joint health in healthy mammals. For example, PCT Application No. PCT/US2021/045853, which was filed on Aug. 13, 2021, and which is incorporated herein by reference, discloses a supplement and method for improving inflammation, joint health, joint mobility, and joint comfort in healthy mammals. In the past, it was believed that the benefits of administering collagen to a mammal were realized at lower dose amounts and that increasing the amount of collagen administered to the mammal had no additional benefits. To the contrary, it has now been discovered that administering greater amounts of collagen to a mammal can unexpectedly and dramatically improve skeletal articulation health in mammals suffering from ailments and diseases such as osteoarthritis and/or skeletal necrosis. In fact, it was discovered that the efficacy of the supplement of the present disclosure continues to increase as the dose of undenatured collagen increases.

For example, in the past, it was widely believed that daily collagen doses of greater than about 0.55, such as greater than about 0.6 mg/kg of body weight per day (e.g. 40 mg of collagen for some mammals such as humans) would have no added beneficial effect on the user or mammal. It was surprisingly and unexpectedly discovered, however, that administering greater than about 0.66 mg/kg of body weight per day of collagen can have dramatic beneficial effects on joint injuries or diseases. In this regard, the method of the present disclosure, in one aspect, can administer greater amounts of collagen to a mammal sufficient to lessen the severity of a skeletal articulation injury or disease as shown through a change in at least one biomarker in comparison to administering less than 0.3 mg/kg of body weight per day of the joint treating composition, such as in comparison to administering less than 0.5 mg/kg of body weight per day of the joint treating composition, such as in comparison to administering less than 0.6 mg/kg of body weight per day of the joint treating composition.

The joint treating composition of the present disclosure, when administered to mammals suffering from ailments or injuries to the skeletal articulations, can dramatically reduce pain in addition to healing the skeletal joints. When administered to mammals suffering from injuries or ailments to one or more skeletal articulations, the joint treating composition can dramatically improve mobility and improve joint and tissue health which can be measured through the Kellgren Lawrence score and the Mankin score. For example, the joint treating composition of the present disclosure can be administered to a mammal over a period of time sufficient to change a Kellgren-Lawrence score of the mammal by at least about 20%. The joint treating composition of the present disclosure can also be administered to a mammal over a period of time sufficient to change a Mankin score of the mammal by at least about 15%.

It was also discovered that administering the joint treating composition to a mammal in therapeutically effective amounts can dramatically impact and improve various biomarkers that are related to skeletal health and/or inflammation. For example, in mammals suffering from disease or injury to a skeletal articulation, the joint treating composition of the present disclosure can dramatically and unexpectedly reduce tumor necrosis factor alpha (TNF a), which is believed to play an important role in inflammation by initiating an inflammatory cascade. The method of the present disclosure, however, can reduce serum TNF a in the body of the mammal by greater than about 5%, such as greater than about 15%, such as greater than about 25%, and generally less than about 120%, such as less than about 80%. Similarly, TNF a protein can be reduced within the body of the mammal by greater than about 10%, such as by greater than about 20%, such as greater than about 25%, such as greater than about 35%, and generally less than about 80%. The above reductions can occur after a period of time of being administered the joint treating composition at least once every three days, such as at least every day. The above results, for instance, can be shown after 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease serum interleukin-1 levels in a mammal suffering from osteoarthritis or other injury or ailment to a skeletal articulation. Serum interleukin levels can decrease by greater than about 10%, such as greater than about 20%, such as greater than about 30%, such as greater than about 40%, and generally less than about 100%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease serum interleukin-6 levels in a mammal suffering from osteoarthritis or other injury or ailment to a skeletal articulation. Serum interleukin levels can decrease by greater than about 10%, such as greater than about 20%, such as greater than about 30%, such as greater than about 40%, and generally less than about 120%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease serum C-reactive protein levels in a mammal suffering from osteoarthritis or other injury or ailment to a skeletal articulation. Serum C-reactive protein levels can decrease by greater than about 10%, such as greater than about 20%, such as greater than about 30%, such as greater than about 40%, and generally less than about 90%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease prostaglandin E2 levels in a mammal suffering from osteoarthritis or other injury or ailment to a skeletal articulation. Prostaglandin E2 levels can decrease by greater than about 8%, such as greater than about 15%, such as greater than about 25%, such as greater than about 30%, and generally less than about 80%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease interleukin-1-B protein levels in a mammal suffering from osteoarthritis or other injury or ailment to a skeletal articulation. Interleukin-1-B protein levels can decrease by greater than about 8%, such as greater than about 10%, such as greater than about 15%, such as greater than about 20%, and generally less than about 60%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease interleukin-6 protein levels in a mammal suffering from osteoarthritis or other injury or ailment to a skeletal articulation. Interleukin-6 protein levels can decrease by greater than about 10%, such as greater than about 15%, such as greater than about 20%, such as greater than about 30%, such as greater than about 40%, and generally less than about 80%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease interleukin-10 protein levels in a mammal suffering from osteoarthritis or other injury or ailment to a skeletal articulation. Interleukin-10 protein levels can decrease by greater than about 10%, such as greater than about 20%, such as greater than about 30%, such as greater than about 40%, and generally less than about 100%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease cyclooxygenase 2 protein levels in a mammal suffering from osteoarthritis or other injury or ailment to a skeletal articulation. Cyclooxygenase 2 protein levels can decrease by greater than about 7%, such as greater than about 10%, such as greater than about 20%, such as greater than about 30%, and generally less than about 90%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease nuclear factor kappa B protein levels in a mammal suffering from osteoarthritis or other injury or ailment to a skeletal articulation. Nuclear factor kappa B protein levels can decrease by greater than about 10%, such as greater than about 20%, such as greater than about 30%, such as greater than about 35%, and generally less than about 80%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease matrix metallopeptidase 3 levels in a mammal suffering from osteoarthritis or other injury or ailment to a skeletal articulation. Matrix metallopeptidase 3 levels can decrease by greater than about 15%, such as greater than about 25%, such as greater than about 35%, and generally less than about 75%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease cartilage oligomeric matrix serum levels in a mammal suffering from osteoarthritis or other injury or ailment to a skeletal articulation. Cartilage oligomeric matrix serum levels can decrease by greater than about 10%, such as greater than about 20%, such as greater than about 30%, and generally less than about 80%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also decrease cartilage oligomeric matrix protein levels in a mammal suffering from osteoarthritis or other injury or ailment to a skeletal articulation. Cartilage oligomeric matrix protein levels can decrease by greater than about 10%, such as greater than about 15%, such as greater than about 25%, such as greater than about 30%, and generally less than about 80%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

The joint treating composition of the present disclosure can also increase transforming growth factor B levels in a mammal suffering from osteoarthritis or other injury or ailment to a skeletal articulation. Transforming growth factor B protein levels can increase by greater than about 5%, such as greater than about 10%, such as greater than about 15%, such as greater than about 20%, and generally less than about 65%. The above effects can occur after treatment with the joint treating composition for a period of time of 20 weeks of treatment, such as after 15 weeks of treatment, such as after 12 weeks of treatment, such as after 10 weeks of treatment, such as after 8 weeks of treatment, such as after 6 weeks of treatment, such as after 4 weeks of treatment.

As shown above, the effect on biomarkers within the mammal can be dramatic and far exceed biomarker influence recorded in the past when a mammal has been administered collagen.

As described above, the joint treating composition of the present disclosure contains collagen alone or in combination with various other additives and ingredients. The joint treating composition may include one or more of any collagen as defined above, and/or, in one aspect, may include one or more of Type I collagen, Type II collagen, Type III collagen, Type IV collagen, or collagen peptides, or a mixture thereof. In one aspect, the joint treating composition contains Type II collagen alone or in combination with one or more of Type I collagen, Type III collagen, Type IV collagen, or collagen peptides. In one aspect, the joint treating composition may include a mixture of Type II collagen (sometimes referred to as native Type II collagen) and undenatured Type II collagen. Additionally or alternatively, the joint treating composition may include a mixture of native Type II collagen and undenatured Type II collagen, in addition to a further collagen, such as Type I, Type III, Type IV, or collagen peptides. Furthermore, in one aspect, the joint treating composition includes whole collagen protein, biologically active peptide fragments of collagen, or a combination thereof.

In one aspect, one or more types of collagen in the joint treating composition may be heat sterilized, such as by autoclaving, and/or may also include salts, such as alkalizing or acid, and/or organic or inorganic salts. Thus, in one aspect, a portion of the collagen may be at least partially hydrolyzed. In one aspect, the collagen is hydrolyzed by any process or compound, including by an acid base agent, an enzyme, heat or other temperature extreme, a chemical, UV, a salt, or combinations thereof. In one aspect, the hydrolyzed collagen is undenatured collagen that has been at least partially hydrolyzed by any method, and in one aspect, may include undenatured collagen that has any portion that has been denatured. However, as discussed, in one aspect, at least a portion of the joint treating composition is undenatured.

As indicated above, in one aspect, the supplement contains a joint treating composition, particularly a Type II collagen composition such as an undenatured Type II collagen composition, an at least partially hydrolyzed Type II collagen composition, or a combination thereof. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® brand from InterHealth Nutraceuticals. UC-II® brand is a natural ingredient that contains a glycosylated, undenatured Type II collagen. The joint treating composition can also comprise a hydrolyzed collagen. The joint treating composition can also comprise a pure protein or active peptide fragments. In one embodiment, the joint treating composition can be free of any bone or bone material. In other embodiments, the joint treating composition can be free of any transforming growth factors (TGFs), bone morphogenetic proteins (BMPs), or both. In still another embodiment, the joint treating composition comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the type II collagen in the tissue, such as low-temperature processing, and formed into doses containing therapeutically effective levels of undenatured type II collagen. Being a natural product some variation from sample to sample is to be expected. These variations can be minimized by blending after comminution. The blending can be aided by analytical techniques which allow the measurement of the amount of undenatured type II collagen and other constituents.

Nonetheless, the present disclosure has found that by carefully forming the particles and sterilizing the type II collagen as discussed above, the undenatured type II collagen may be resistant to gastric acid and digestive enzymes in the stomach. Due to this sterilization process, the undenatured type II collagen also retains its 3-dimensional shape, preserving the bioactive epitope regions. Without wishing to be bound by theory, it is believed that the epitope regions contain the ability to induce oral tolerance as discussed above. Particularly epitope regions allow undenatured collagen to bind to the Peyer's Patches, which have the ability to induce oral tolerance processes.

The amount of the joint treating composition administered to a mammal, the number of doses per unit time, and the length of the treatment can all vary depending upon various factors including the type of mammal being treated, the injury or ailment that is being treated, and the like. In one embodiment, for instance, the joint treating composition is administered to the mammal such that the mammal receives collagen in an amount of at least 0.66 mg/kg of body weight per day. For example, a therapeutically effective amount may include administering collagen to the mammal in an amount greater than about 1 mg/kg of body weight per day, such as greater than about 1.2 mg/kg of body weight per day, such as greater than about 1.4 mg/kg of body weight per day, such as greater than about 1.6 mg/kg of body weight per day, such as greater than about 1.8 mg/kg of body weight per day, such as greater than about 2 mg/kg of body weight per day, such as greater than about 2.2 mg/kg of body weight per day, such as greater than about 2.4 mg/kg of body weight per day, such as greater than about 2.6 mg/kg of body weight per day, and generally less than about 5 mg/kg of body weight per day, such as less than about 4 mg/kg of body weight per day.

In one particular embodiment, collagen is administered to the mammal in an amount of from about 1.2 mg/kg of body weight per day to about 3 mg/kg of body weight per day.

When administering collagen in accordance with the present disclosure to larger mammals including humans, collagen can be administered to the mammal in an amount greater than about 50 mg per day, such as in an amount greater than about 80 mg per day, such as in an amount greater than about 120 mg per day, such as in an amount greater than about 160 mg per day, such as in an amount greater than about 320 mg per day, such as in an amount greater than about 480 mg per day, such as in an amount greater than about 640 mg per day, such as in an amount greater than about 800 mg per day. The amount of collagen administered to the mammal is generally less than about 5,000 mg per day, such as less than about 3,000 mg per day, such as less than about 2,000 mg per day, such as less than about 1,000 mg per day, such as less than about 500 mg per day.

The joint treating composition can be administered to the mammal orally and can be in the form of a nutraceutical supplement. The supplement can be in the form of individual dosage vessels that can be swallowed, chewed, drank, or the like. The dosage vessel can be a capsule or a tablet. The amount of collagen in each dosage vessel, in one embodiment, can be from about 40 mg to about 200 mg. In one aspect, each dosage vessel as described above can be administered to the mammal at least twice daily, such as at least three times daily, such as at least four times daily, and generally less than about eight times daily, such as less than about six times daily, such as less than about five times daily.

In one aspect, undenatured Type II collagen may form all, or substantially all, of the total Type II collagen in the collagen composition, and therefore, may be present in the supplement in the above discussed amounts. However, in one aspect, undenatured Type II collagen may account for about 0.5% to about 95% of the total Type II collagen and/or collagen composition, such as about 1% to about 75%, such as about 1.5% to about 50%, such as about 2% to about 40%, such as about 2.5% to about 15% of the total Type II collagen or total collagen composition, or any ranges or values therebetween.

In one aspect, the collagen composition may further include a preservative salt, such as potassium chloride. Thus, in one aspect, the total amounts of collagen composition discussed above may include Type II collagen and/or undenatured Type II collagen, alone or in combination with a further collagen, a preservative salt, or combinations thereof. In such as aspect, the total Type II collagen, including native and undenatured Type II collagen, may account for about 1% to about 99% of the collagen composition, such as about 2.5% to about 90%, such as about 5% to about 80%, such as about 7.5% to about 70%, such as about 10% to about 60%, such as about 15% to about 50%, such as about 20% to about 35%, or any ranges or values therebetween.

When the Type II collagen includes undenatured Type II collagen, the undenatured Type II collagen may have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′ azobis (2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, a collagen composition having an undenatured Type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, a collagen composition having an undenatured Type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, a collagen composition having an undenatured Type II collagen according to the present disclosure may have a ORAC against hydroxyl radicals of about 10 μmol TE/g or greater, such as about 15 μmol TE/g or greater, such as about 20 μmol TE/g or greater, such as about 25 μmol TE/g or greater, such as about 27.5 μmol TE/g or greater, such as about 30 μmol TE/g or greater, up to about 40 μmol TE/g, or any ranges or values therebetween.

Additionally or alternatively, in one aspect, a collagen composition having an undenatured Type II collagen according to the present disclosure may have a ORAC against peroxynitrite of about 0.5 μmol TE/g or greater, such as about 1 μmol TE/g or greater, such as about 1.5 μmol TE/g or greater, such as about 2 μmol TE/g or greater, such as about 2.25 μmol TE/g or greater, up to about 5 μmol TE/g, or any ranges or values therebetween.

In one aspect, a collagen composition having an undenatured Type II collagen according to the present disclosure may have a ORAC against singlet oxygen of about 500 μmol TE/g or greater, such as about 550 μmol TE/g or greater, such as about 600 μmol TE/g or greater, such as about 650 μmol TE/g or greater, such as about 700 μmol TE/g or greater, such as about 725 μmol TE/g or greater, up to about 1000 μmol TE/g, or any ranges or values therebetween.

Furthermore, in one aspect, a collagen composition having an undenatured Type II collagen according to the present disclosure may have a ORAC against hypochlorite of about 25 μmol TE/g or greater, such as about 30 μmol TE/g or greater, such as about 35 μmol TE/g or greater, such as about 40 μmol TE/g or greater, such as about 45 μmol TE/g or greater, such as up to about 50 μmol TE/g or greater, up to about 75 μmol TE/g, or any ranges or values therebetween.

Furthermore, in one aspect, when the Type II collagen includes undenatured Type II collagen, the undenatured Type II collagen may have a molecular weight of about 10,000 Daltons or more, such as about 15,000 Daltons or more, such as about 20,000 Daltons or more, such as about 25,000 Daltons or more, such as about 30,000 Daltons or more, such as about 35,000 Daltons or more, such as about 40,000 Daltons or more, such as about 45,000 Daltons or more, such as about 50,000 Daltons or more, such as about 55,000 Daltons or more, such as about 60,000 Daltons or more, such as about 65,000 Daltons or more, such as about 70,000 Daltons or more, such as about 75,000 Daltons or more, such as about 80,000 Daltons or more, such as about 85,000 Daltons or more, such as about 90,000 Daltons or more such as about 95,000 Daltons or more, such as about 100,000 Daltons or more, up to about 350,000 Daltons or less, or any ranges or values therebetween.

While various aspects and benefits have been discussed, in one aspect, the collagen composition is incorporated into a suitable delivery form prior to incorporation into a dosage form as discussed below. In one aspect, the composition of the present disclosure may be included as a capsule or a tablet.

Alternatively, the composition can be an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the Type II collagen), and the emulsion may be used to incorporate the composition into a water-based application. However, it should be understood that, in one aspect, a water-soluble Type II collagen may be used in the supplement.

The oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible.

The oil-in-water emulsion may also contain water. In certain aspects, the oil-in-water emulsion may contain from about 5% to 35% by weight of water.

In some aspects, the oil-in-water emulsion may contain one or more stabilizers or suspension promoting agents. For example, in certain aspects, the oil-in-water emulsion may contain one or more gums, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight. An example of a stabilizer is silica which can be added in an amount of less than about 2% by weight, such as less than about 1.5% by weight, such as less than about 1% by weight, such as less than about 0.5% by weight.

In some aspects, the oil-in-water emulsion disclosed herein may be used any suitable dosage form, such as tablets, gummy chewables, edible films, lozenges, liquid suspensions, syrups, lipid micelles, spray-dried dispersions, nanoparticles, and the like, which may also be incorporated into a further supplement.

Alternatively, the oil-in-water emulsion may be contained in a nutritional product, such as a supplement, a food product, or in a beverage. For example, in certain aspects, the oil-in-water emulsion may be incorporated into a liquid nutritional product, such as a nutritional supplement or infant formula, to be consumed by a mammal. Furthermore, the oil-in-water emulsions provided herein may be added to any liquid nutritional product designed to provide nutritional supplementation to a mammal.

The nutritional product can include any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, powder, or the like.

The supplement according to the present disclosure may be administered to the mammal including by oral, enteral or by-inhalation administration of whole collagen protein or biologically active peptide fragments of collagen. For instance, in one aspect, it is believed that the whole collagen protein or biologically active peptide fragments of collagen enhances the content of trans-L-hydroxyproline based on the total weight of amino acids contained in collagen type II, and enables efficient production of trans-L-hydroxyproline to enhance the efficacy.

The supplement composition of the present disclosure may further comprise one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, fats, oils and/or waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the supplement composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the supplement composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the supplement composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company. In an alternative embodiment, the polymer binder may comprise arabinogalactan.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the supplement composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Furthermore, in one aspect, the supplement may be formulated into a food and/or supplement for exercise, sport, or daily nutritional purposes. In such an aspect, the supplement may further include at least one vitamin, such as at least one of vitamin B, vitamin C, and vitamin E. Vitamins may be contained in the supplement in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in supplement in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in supplement in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the supplement contains at least one mineral, such as at least one of potassium magnesium, zinc, or calcium. Minerals may be contained in the supplement in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the supplement contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the supplement contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the supplement contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Thus, in one aspect, the supplement according to the present disclosure may also include one or more additional joint supplements such as hydroxy citric acid, glucosamine, chondroitin, methylsulfonylmethane, eggshell membrane, green lipped muscle, or the like, or combinations thereof, and/or an enhancer of collagen absorption, such as vitamin c, in addition to the undenatured collagen.

Moreover, the supplement may be suitable for administration to any mammal. For instance, the mammal may be human or canine. The composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood.

Nonetheless, certain embodiments of the present disclosure may be better understood according to the following examples, which are intended to be non-limiting and exemplary in nature.

### Example 1

The following example demonstrates some of the benefits and advantages of the present disclosure. In this example, Wistar rats were collected and placed into groups of seven. Older rats, 18 to 20 months old, were tested separately from younger rats that were 8 weeks old. Except for an older rat control group and a younger rat control group, the rats were intra-articularly injected with monosodium iodoacetate (“MIA”) in order to simulate a joint ailment, such as osteoarthritis.

Except for the control groups and an older rat group and a younger rat group that were only administered the MIA, the remaining rat groups were administered an joint treating composition in accordance with the present disclosure varying the amount of collagen from group to group. The collagen used in the experiments was an undenatured Type II collagen referred to as “UCII”. More particularly, the following rat groups were tested:

| Older Rat Groups | Younger Rat Groups |
|---|---|
| Control | Control |
| 1 mg MIA | 1 mg MIA |
| 1 mg MIA + UCII 0.66 mg/kg of | 1 mg MIA + UCII 0.66 mg/kg of |
| body weight | body weight |
| 1 mg MIA + UCII 1.33 mg/kg of | 1 mg MIA + UCII 1.33 mg/kg of |
| body weight | body weight |
| 1 mg MIA + UCII 2 mg/kg of | 1 mg MIA + UCII 2 mg/kg of |
| body weight | body weight |

As described above, each group was made up of seven rats. Tests were conducted on the rats after 3 days, after 7 days, after 14 days, and after 28 days.

The following tests were conducted:

| Details | Old Rats | Young Rats |
|---|---|---|
| Quantitative measures | Stride length/paw area/paw | Stride length/paw area/paw |
| | width | width |
| OA measures | Joint diameter/Kellgren | Joint diameter/Kellgren |
| | score/Mankin screen | score/Mankin screen |
| Serum | IL 1-β, IL-6, TNF-α, COMP, | IL 1-β, IL-6, TNF-α, COMP, |
| | CRP and Prostaglandin E2; | CRP and Prostaglandin E2; |
| | Safety Measures | Safety Measures |
| Gene proteins/expression | IL 1-β, IL-6, IL 10, TNF-α, | IL 1-β, IL-6, IL 10, TNF-α, |
| | COX2, NFkβ Collagen Type | COX2, NFkβ Collagen Type |
| | 2, MMP3, COMP and TGF-β | 2, MMP3, COMP and TGF-β |

The measurements of paw area, stride length and paw width on days 3, 7, 14 and 28 in MIA induced osteoarthritic rats are shown below:

Dose response observed at Day 14 in older rats over controls

| | | Old Rats - Groups | | | | |
|---|---|---|---|---|---|---|
| | | | | MIA + UCII | MIA + UCII | MIA + UCII |
| Items | | Control | MIA | 0.66 | 1.33 | 2.00 |
| Paw Area, cm² | | | | | | |
| | Day 3 | 5.04 ± 0.26 | 5.07 ± 0.28 | 5.06 ± 0.22 | 5.06 ± 0.22 | 5.14 ± 0.24 |
| | Day 7 | 5.11 ± 0.25 | 5.03 ± 0.31 | 5.01 ± 0.24 | 5.05 ± 0.31 | 5.16 ± 0.21 |
| | Day 14 | 5.40 ± 0.23^{a} | 4.16 ± 0.22^{d} | 4.55 ± 0.22^{cd} | 4.73 ± 0.43^{bc} | 5.07 ± 0.14^{ab} |
| | Day 28 | 5.44 ± 0.16^{a} | 4.11 ± 0.45^{d} | 4.57 ± 0.21^{c} | 4.87 ± 0.17^{bc} | 5.23 ± 0.12^{ab} |
| Stride Length, cm | | | | | | |
| | Day 3 | 7.73 ± 0.50 | 7.70 ± 0.38 | 7.82 ± 0.47 | 7.76 ± 0.24 | 7.76 ± 0.47 |
| | Day 7 | 7.98 ± 0.53 | 7.41 ± 0.24 | 7.42 ± 0.75 | 7.47 ± 0.44 | 7.57 ± 0.27 |
| | Day 14 | 8.66 ± 0.40^{a} | 6.70 ± 0.16^{c} | 7.30 ± 0.79^{bc} | 7.76 ± 0.28^{b} | 8.01 ± 0.49^{ab} |
| | Day 28 | 8.87 ± 0.25^{a} | 6.82 ± 0.11^{c} | 7.32 ± 0.27^{d} | 7.85 ± 0.14^{c} | 8.42 ± 0.19^{b} |
| Paw Width, cm | | | | | | |
| | Day 3 | 1.71 ± 0.05 | 1.71 ± 0.09 | 1.69 ± 0.04 | 1.71 ± 0.04 | 1.71 ± 0.06 |
| | Day 7 | 1.76 ± 0.05 | 1.69 ± 0.05 | 1.74 ± 0.04 | 1.73 ± 0.06 | 1.74 ± 0.09 |
| | Day 14 | 1.96 ± 0.03^{a} | 1.62 ± 0.09^{c} | 1.78 ± 0.10^{b} | 1.83 ± 0.05^{ab} | 1.90 ± 0.10^{ab} |
| | Day 28 | 1.96 ± 0.07^{a} | 1.64 ± 0.05^{d} | 1.80 ± 0.04^{c} | 1.85 ± 0.09^{bc} | 1.92 ± 0.03^{ab} |

Dose response observed at Day 7 in young adult rats over controls

| | | Young Rats - Groups | | | | |
|---|---|---|---|---|---|---|
| | | | | MIA + UCII | MIA + UCII | MIA + UCII |
| Items | | Control | MIA | 0.66 | 1.33 | 2.00 |
| Paw Area, cm² | | | | | | |
| | Day 3 | 4.13 ± 0.21 | 4.11 ± 0.21 | 4.14 ± 0.18 | 4.16 ± 0.22 | 4.17 ± 0.18 |
| | Day 7 | 4.26 ± 0.09^{a} | 3.87 ± 0.10^{b} | 4.05 ± 0.31^{ab} | 4.11 ± 0.31^{ab} | 4.11 ± 0.20^{ab} |
| | Day 14 | 4.25 ± 0.10^{a} | 3.30 ± 0.35^{d} | 3.52 ± 0.18^{cd} | 3.74 ± 0.32^{bc} | 3.94 ± 0.29^{ab} |
| | Day 28 | 4.28 ± 0.22^{a} | 3.26 ± 0.28^{d} | 3.54 ± 0.15^{cd} | 3.84 ± 0.25^{bc} | 3.99 ± 0.11^{ab} |
| Stride Length, cm | | | | | | |
| | Day 3 | 6.88 ± 0.53 | 6.82 ± 0.63 | 6.99 ± 0.65 | 6.94 ± 0.60 | 6.84 ± 0.61 |
| | Day 7 | 7.12 ± 0.66^{a} | 6.11 ± 0.36^{b} | 6.42 ± 0.42^{ab} | 6.75 ± 0.43^{ab} | 6.77 ± 0.30^{ab} |
| | Day 14 | 7.62 ± 0.39^{a} | 5.94 ± 0.30^{c} | 6.37 ± 0.90^{bc} | 6.75 ± 0.75^{abc} | 7.05 ± 0.38^{ab} |
| | Day 28 | 7.65 ± 0.07^{a} | 6.04 ± 0.20^{d} | 6.57 ± 0.28^{c} | 6.95 ± 0.38^{b} | 7.25 ± 0.07^{b} |
| Paw Width, cm | | | | | | |
| | Day 3 | 1.51 ± 0.02 | 1.48 ± 0.06 | 1.48 ± 0.08 | 1.49 ± 0.06 | 1.49 ± 0.04 |
| | Day 7 | 1.63 ± 0.05^{a} | 1.47 ± 0.05^{b} | 1.54 ± 0.08^{b} | 1.54 ± 0.06^{b} | 1.55 ± 0.03^{ab} |
| | Day 14 | 1.66 ± 0.04^{a} | 1.50 ± 0.05^{c} | 1.56 ± 0.06^{bc} | 1.57 ± 0.06^{bc} | 1.59 ± 0.04^{ab} |
| | Day 28 | 1.66 ± 0.09^{a} | 1.48 ± 0.07^{b} | 1.55 ± 0.09^{ab} | 1.59 ± 0.04^{a} | 1.60 ± 0.05^{a} |

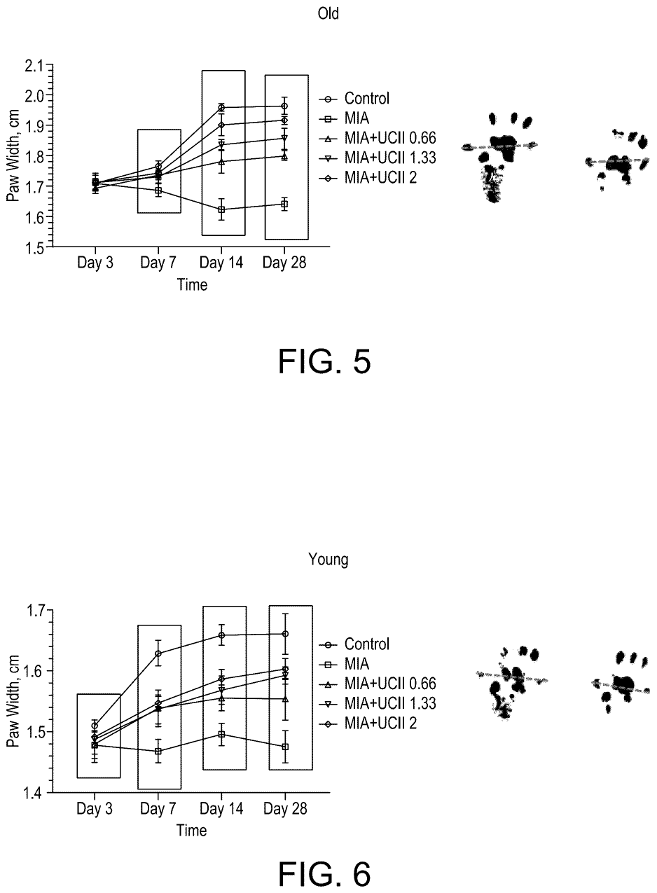

The results obtained in the above tables are also illustrated in FIGS. 5-6. As shown, the joint treating composition of the present disclosure dramatically and unexpectedly improved the mobility of the MIA induced rats.

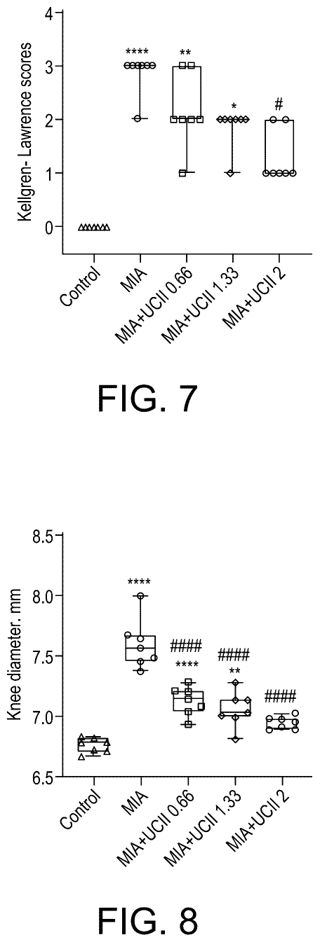

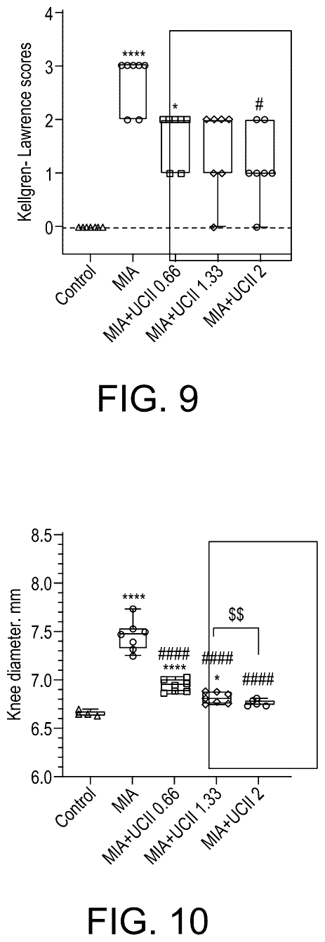

The rats were also tested for Kellgren Lawrence Score and knee diameter after 28 days. The results are illustrated in FIGS. 7-10.

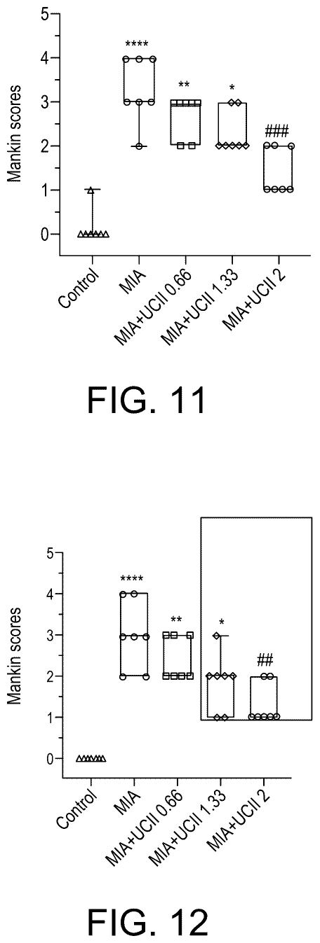

The rat groups were also tested for Mankin Score which is directed to reducing the grade of lesion score in knee diameter. The results are illustrated in FIGS. 11-12.

The following is a summary of changes in the Kellgren Lawrence Score and the Mankin Score:

### Dose Response Study—Young Rats

| | Joint | Kellgren- | |
|---|---|---|---|
| Dose | diameter, mm | Lawrence scores | Mankin Score |
| mg/kg BW | % change from MIA group | | |
| MIA + UCII 0.66 | 6.8992 | 36.84 | 19.0476 |
| MIA + UCII 1.33 | 8.586 | 47.3684 | 38.0952 |
| MIA + UCII 2 | 9.295 | 57.8947 | 57.1429 |

### Dose Response Study—Old Rats

| | Joint | Kellgren- | |
|---|---|---|---|
| Dose | diameter, mm | Lawrence scores | Mankin Score |
| mg/kg BW | % change from MIA group | | |
| MIA + UCII 0.66 | 6.170 | 25.000 | 17.3913 |
| MIA + UCII 1.33 | 7.148 | 35.000 | 30.43478 |
| MIA + UCII 2 | 8.578 | 55.000 | 56.52174 |

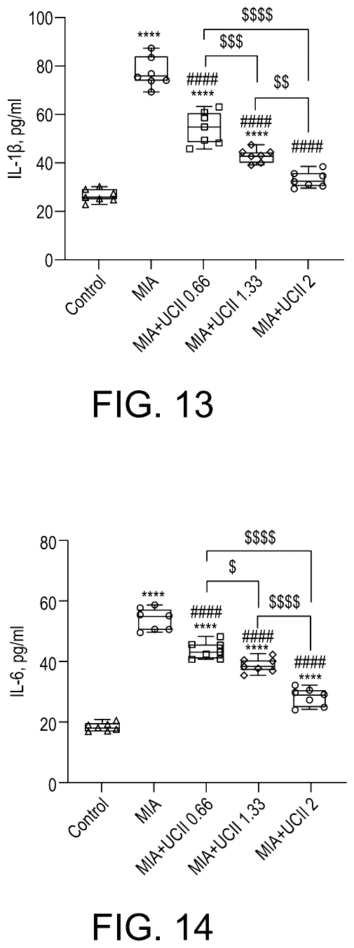

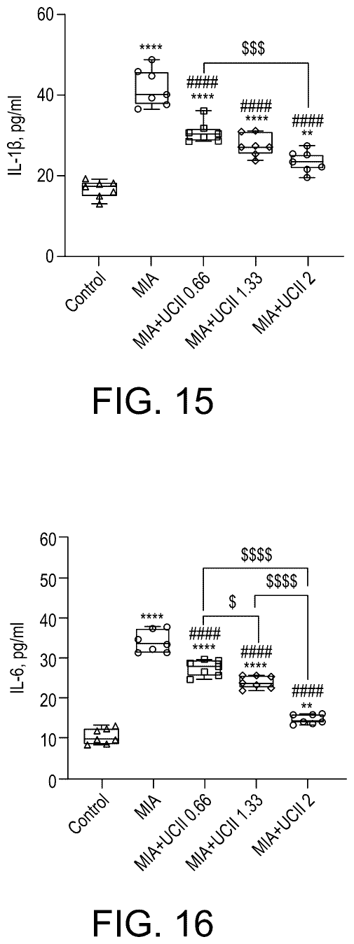

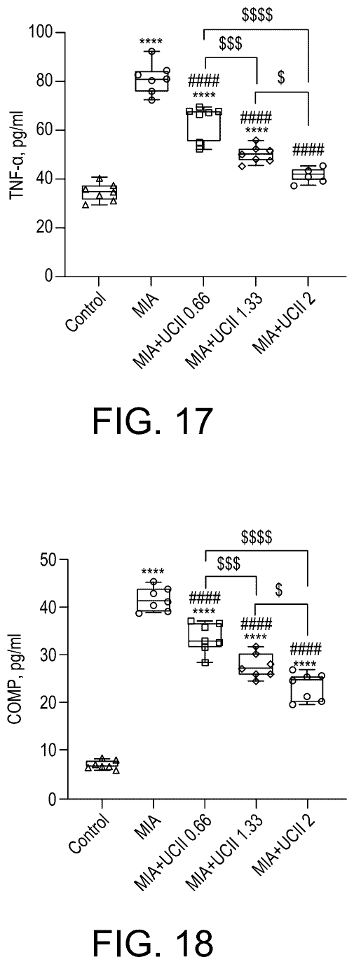

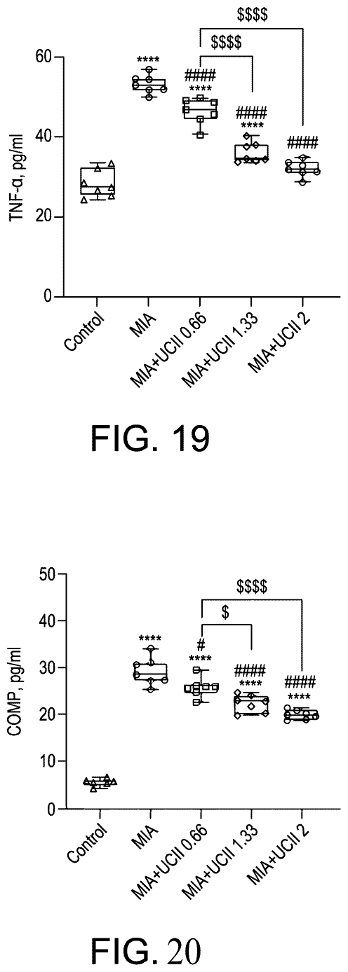

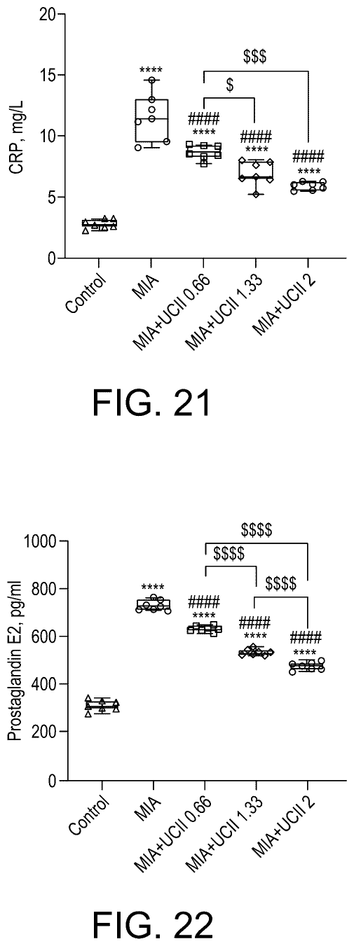

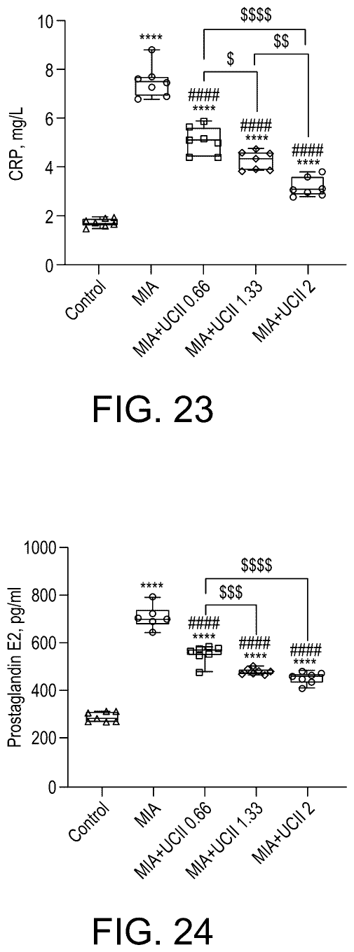

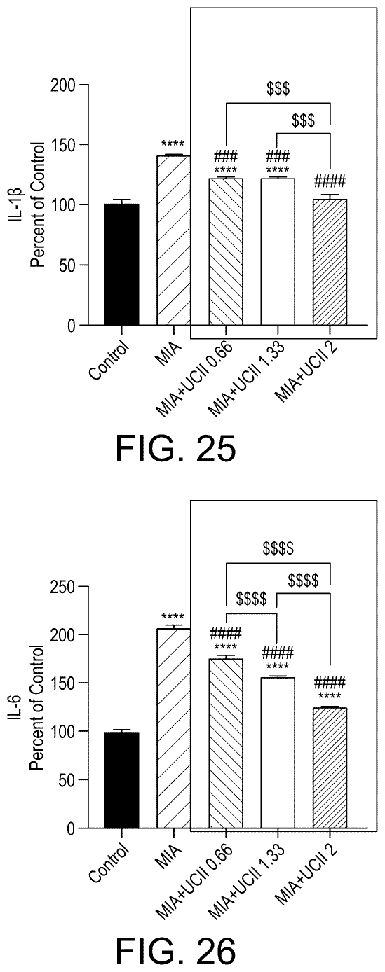

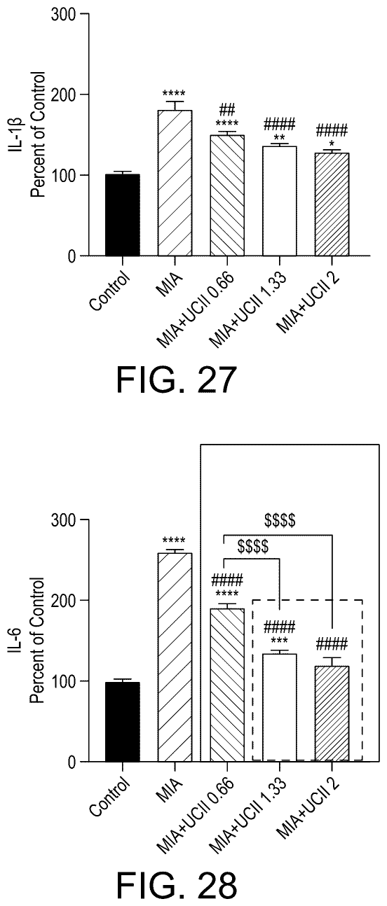

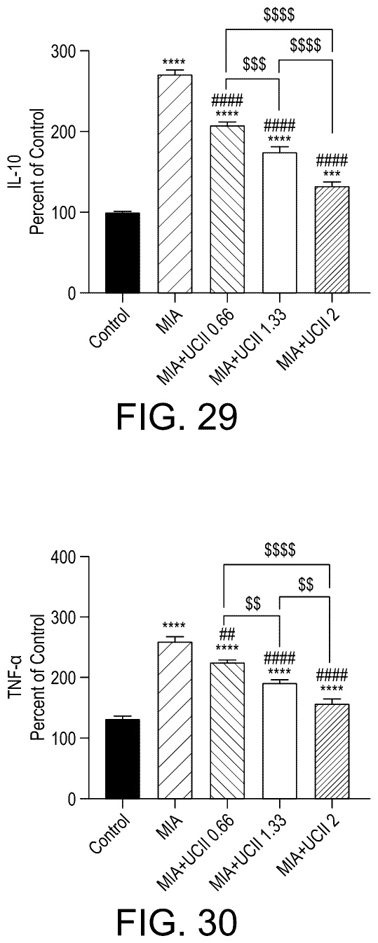

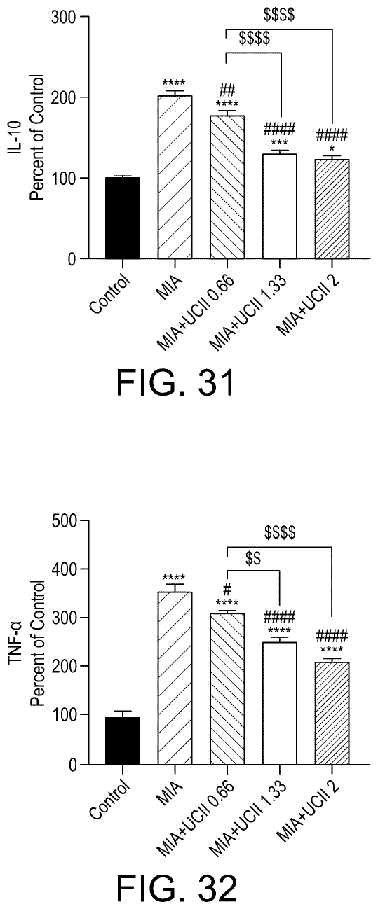

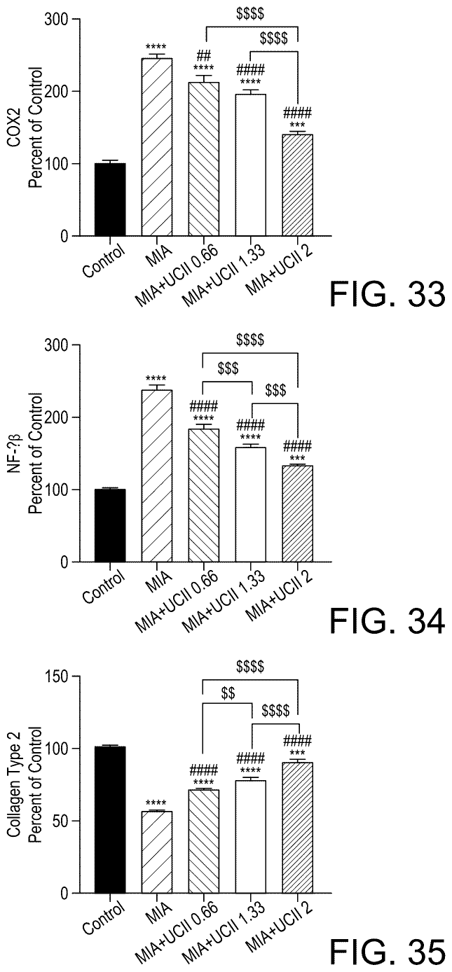

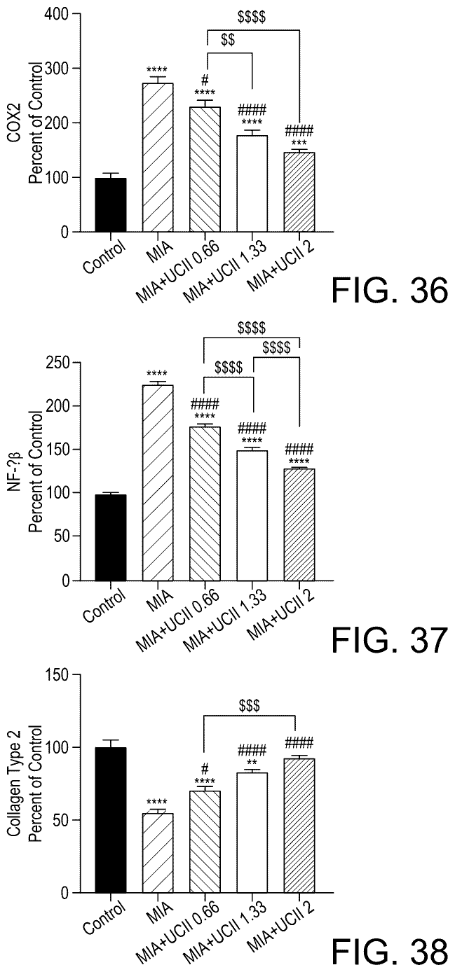

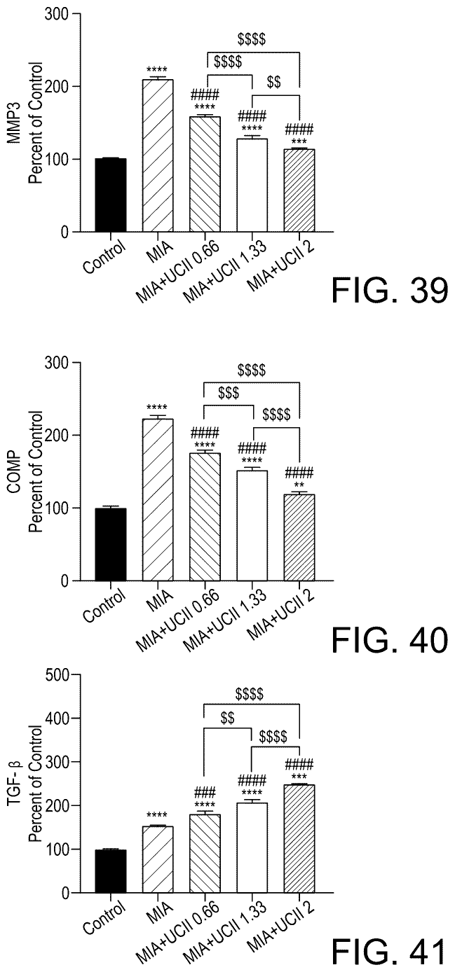

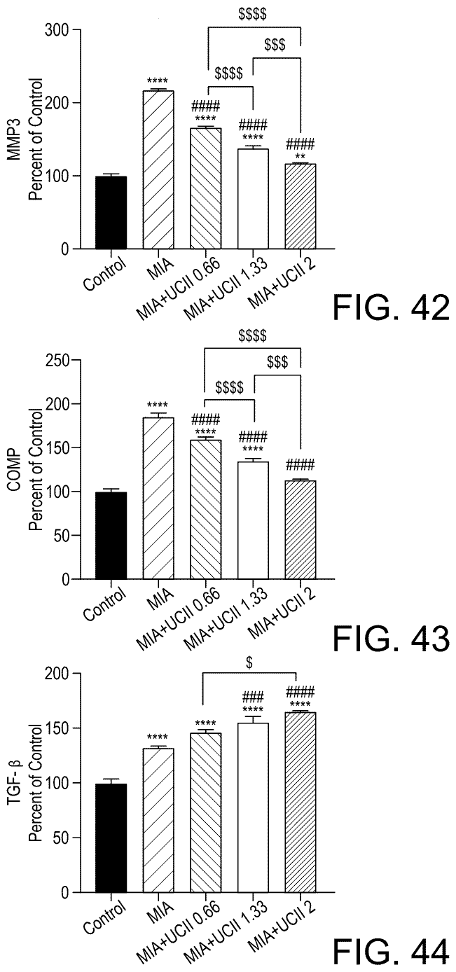

After 28 days, various biomarkers comprising serum levels and protein levels were tested. The results are illustrated in FIGS. 13-44.

Various different safety markers were also tested to show that there was no significant difference between treatments. Those results are as follows:

| | Old Groups | | | | |
|---|---|---|---|---|---|
| | | | MIA + | MIA + | MIA + |
| Items | Control | MIA | UCII 0.66 | UCII 1.33 | UCII 2.00 |
| Glucose, mg/dL | 114.43 ± 1.69 | 116.43 ± 3.33 | 115.14 ± 2.78 | 116.14 ± 5.34 | 114.57 ± 2.79 |
| BUN, g/dL | 21.57 ± 0.45 | 21.11 ± 0.61 | 21.67 ± 0.47 | 21.10 ± 0.68 | 21.04 ± 0.34 |
| Creatine, mg/dL | 0.46 ± 0.02 | 0.50 ± 0.01 | 0.48 ± 0.02 | 0.49 ± 0.03 | 0.46 ± 0.01 |
| ALT, U/L | 118.57 ± 3.85 | 117.71 ± 5.47 | 115.00 ± 5.91 | 120.43 ± 3.80 | 117.29 ± 4.72 |
| AST, U/L | 129.71 ± 4.52 | 134.70 ± 5.19 | 130.69 ± 7.07 | 133.69 ± 5.93 | 131.77 ± 4.54 |

BUN: Blood urea nitrogen; ALT: Alanine aminotransferase; AST: Aspartate aminotransferase. Mean values of items are demonstrated with ±standard error of mean.

| | Young Groups | | | | |
|---|---|---|---|---|---|
| | | | MIA + | MIA + | MIA + |
| Items | Control | MIA | UCII 0.66 | UCII 1.33 | UCII 2 |
| Glucose, mg/dL | 114.71 ± 2.39 | 115.57 ± 3.04 | 116.00 ± 2.33 | 115.71 ± 2.66 | 116.29 ± 3.03 |
| BUN, g/dL | 20.07 ± 0.28 | 20.41 ± 0.56 | 20.74 ± 0.79 | 20.89 ± 0.71 | 20.21 ± 0.73 |
| Creatine, mg/dL | 0.37 ± 0.02 | 0.36 ± 0.02 | 0.37 ± 0.02 | 0.36 ± 0.03 | 0.37 ± 0.03 |
| ALT, U/L | 109.71 ± 4.14 | 111.86 ± 2.99 | 109.29 ± 4.26 | 107.86 ± 3.02 | 110.29 ± 7.17 |
| AST, U/L | 117.86 ± 4.62 | 120.57 ± 4.08 | 119.86 ± 4.45 | 121.29 ± 5.18 | 118.86 ± 6.51 |
| TP, g/dL | 8.19 ± 0.08 | 8.23 ± 0.20 | 8.10 ± 0.12 | 8.43 ± 0.18 | 8.50 ± 0.12 |
| Albumin, g/dL | 3.74 ± 0.09 | 3.79 ± 0.14 | 3.76 ± 0.08 | 3.73 ± 0.09 | 3.70 ± 0.13 |

BUN: Blood urea nitrogen; ALT: Alanine aminotransferase; AST: Aspartate aminotransferase; TP: Total protein. Mean values of items are demonstrated with ±standard error of mean.

As shown in the attached figures, the rats administered with the joint treating composition of the present disclosure displayed dramatic improvement in mobility. There was a significant dose response for the older rats on day 14 and for the younger rats on day 7. In addition, anti-inflammatory properties were observed. The anti-inflammatory properties were observed in the younger rats only after 3 days. It was discovered that a higher dose of collagen produced significant effects regarding the anti-inflammatory properties.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method of treating a skeletal articulation injury or disease in a mammal comprising:
supplying to the mammal a nutraceutical supplement containing a therapeutically effective amount of a joint treating composition,
the joint treating composition comprising collagen,
the joint treating composition being administered in doses, and
wherein the therapeutically effective amount is an amount of at least about 0.66 mg/kg of body weight per day and sufficient to lessen the severity the skeletal articulation injury or disease as shown through a change in at least one joint health marker in comparison to administering less than 0.3 mg/kg of body weight per day of the joint treating composition.

2. A method as defined in claim 1, wherein the therapeutically effective amount of the joint treating composition administered to the mammal includes a collagen amount of at least 1 mg/kg of body weight per day.

3. A method as defined in claim 1, wherein the therapeutically effective amount of the joint treating composition administered to the mammal includes a collagen amount of at least 2 mg/kg of body weight per day.

4. A method as defined in claim 1, wherein the therapeutically effective amount of the joint treating composition administered to the mammal includes a collagen amount of at least 1.2 mg/kg of body weight per day to about 3 mg/kg of body weight per day.

5. A method as defined in claim 1, wherein the collagen comprises undenatured Type II collagen.

6. A method as defined in claim 1, wherein the joint treating composition is administered to the mammal in a therapeutically effective amount sufficient to decrease serum tumor necrosis factor by at least about 5% and generally less than about 80% after 28 days of treatment.

7. A method as defined in claim 1, wherein the joint treating composition is administered to the mammal in a therapeutically effective amount sufficient to decrease serum C-reactive protein by at least about 10% and generally less than about 90% after 28 days of treatment.

8. A method as defined in claim 1, wherein the joint treating composition is administered to the mammal in a therapeutically effective amount sufficient to decrease prostaglandin E2 by at least about 8% and generally less than about 80% after 28 days of treatment.

9. A method as defined in claim 1, wherein the joint treating composition is administered to the mammal in a therapeutically effective amount sufficient to decrease interleukin-10 protein by at least about 10% and generally less than about 100% after 28 days of treatment.

10. A method as defined in claim 1, wherein the joint treating composition is administered to the mammal in a therapeutically effective amount sufficient to decrease tumor necrosis factor by at least about 10% and generally less than about 80% after 28 days of treatment.

11. A method as defined in claim 1, wherein the joint treating composition is administered to the mammal in a therapeutically effective amount sufficient to decrease cyclooxygenase 2 protein by at least about 7% and generally less than about 90% after 28 days of treatment.

12. A method as defined in claim 1, wherein the joint treating composition is administered to the mammal in a therapeutically effective amount sufficient to decrease nuclear factor kappa B by at least about 10% and generally less than about 80% after 28 days of treatment.

13. A method as defined in claim 1, wherein the joint treating composition is administered to the mammal in a therapeutically effective amount sufficient to decrease matrix metallopeptidase 3 by at least about 15% and generally less than about 75% after 28 days of treatment.

14. A method as defined in claim 1, wherein the joint treating composition is administered to the mammal in a therapeutically effective amount sufficient to increase transforming growth factor B protein by at least about 5% and generally less than about 80% after 28 days of treatment.

15. A method as defined in claim 1, wherein the joint treating composition is administered to the mammal in a therapeutically effective amount sufficient to decrease serum cartilage oligomeric matrix by at least about 10% and generally less than about 80% after 28 days of treatment.

16. A method as defined in claim 1, wherein the joint treating composition is administered to the mammal in a therapeutically effective amount sufficient to decrease cartilage oligomeric matrix protein by at least about 10% and generally less than about 80% after 28 days of treatment.

17. A method as defined in claim 1, wherein the joint treating composition is administered to the mammal in a therapeutically effective amount sufficient to decrease serum interleukin-1 by at least about 10% and generally less than about 100% after 28 days of treatment.

18. A method as defined in claim 1, wherein the joint treating composition is administered to the mammal in a therapeutically effective amount sufficient to decrease serum interleukin-6 by at least about 10% and generally less than about 120% after 28 days of treatment.

19. A method as defined in claim 1, wherein the joint treating composition is administered to the mammal in a therapeutically effective amount sufficient to decrease interleukin-1-B protein by at least about 8% and generally less than about 60% after 28 days of treatment.

20. A method as defined in claim 1, wherein the joint treating composition is administered to the mammal in a therapeutically effective amount sufficient to decrease interleukin-6 protein by at least about 10% and generally less than about 80% after 28 days of treatment.

21. A method as defined in claim 1, wherein the joint treating composition is administered to the mammal in a therapeutically effective amount sufficient to change a Kellgren-Lawrence score of the mammal by at least about 20%.

22. A method as defined in claim 1, wherein the joint treating composition is administered to the mammal in a therapeutically effective amount sufficient to change a Mankin score of the mammal by at least about 15%.

23. A method as defined in claim 1, wherein collagen is administered to the mammal in an amount greater than 50 mg per day and generally less than about 800 mg per day.
